(19) United States
(12) Patent Application Publication
(10) Pub. No.: US 2021/0393149 A1
(43) Pub. Date: Dec. 23, 2021

(54) METHODS FOR DETERMINING BLOOD OXYGENATION AND TISSUE PERFUSION LEVELS AND DEVICES THEREOF

(71) Applicant: CHEMIMAGE CORPORATION, Pittsburgh, PA (US)

(72) Inventors: Patrick J. TREADO, Pittsburgh, PA (US); Heather E. GOMER, Sewickley, PA (US); Shona STEWART, Pittsburgh, PA (US); Aaron G. SMITH, Monroeville, PA (US)

(21) Appl. No.: 17/355,933

(22) Filed: Jun. 23, 2021

Related U.S. Application Data
(60) Provisional application No. 63/042,897, filed on Jun. 23, 2020.

(51) Int. Cl. A61B 5/026

(52) U.S. Cl. CPC A61B 5/0261

(57) ABSTRACT

Methods for improved tissue perfusion monitoring are disclosed. A method includes collecting hyperspectral image data from an image sensor positioned to collect interacted photons from a tissue region resulting from illumination of the tissue sample at a plurality of wavelengths in the visible, near infrared, or shortwave infrared regions. Hypercubes are generated based on the collected hyperspectral image data. The hypercubes are analyzed to identify one or more of the plurality of wavelengths resulting in contrast in the hyperspectral images. One or more regions in the tissue region with altered perfusion states are identified based on the contrast in the hyperspectral images. A tissue perfusion monitoring computing device and non-transitory medium are also disclosed.

### CROSS-REFERENCE TO RELATED APPLICATIONS

This application claims the benefit of U.S. Provisional Patent Application No. 63/042,897 filed Jun. 23, 2020, the entirety of which is incorporated by reference herein.

### TECHNICAL FIELD

The present disclosure relates generally to systems and methods for improved detection of blood oxygenation and tissue perfusion levels. In particular, the present disclosure relates to systems and methods that detect blood oxygenation and tissue perfusion using optical devices.

### BACKGROUND

Blood oxygenation and tissue perfusion are important metrics that may be monitored for a patient in a clinical or surgical setting. A patient's blood oxygenation level is the ratio of oxygen-saturated hemoglobin and total hemoglobin available for the patient. Blood oxygenation is currently monitored through arterial blood gas analysis or using pulse oximeters. Arterial blood gas analysis requires an invasive arterial blood draw for sample collection and subsequent processing of the sample for accurate diagnosis. Pulse oximeters are placed in contact with the patient, most commonly on a finger but also on a toe or an ear, in order to measure blood oxygenation levels. Pulse oximeters are only accurate to within plus or minus 2% of blood draw measurements, and are not useful for measuring tissue perfusion during a surgery.

Tissue perfusion refers to the passage of blood through tissue via vessels including veins, arteries, and capillaries. Hypoperfusion is a state of decreased tissue perfusion and represents a significant risk to surgical patients. Successful hemodynamic protocols that maintain adequate tissue perfusion during surgery lead to reduced mortality and postoperative organ failure in high-risk patients. Thus, there is a need for a real-time, reagentless, and non-contact method for monitoring tissue perfusion during surgical procedures in order to increase the effectiveness of hemodynamic protocols.

The present disclosure is directed to this and other advantageous improvements in blood oxygenation and tissue perfusion detection.

### SUMMARY

In one embodiment, there is a method of detecting tissue perfusion, the method comprising: collecting, by a tissue perfusion monitoring computing device, image data from an image sensor positioned to collect interacted photons from a tissue region resulting from illumination of the tissue sample at a plurality of wavelengths; analyzing, by the tissue perfusion monitoring computing device, the image data to identify one or more of the plurality of wavelengths resulting in contrast in the image data; and identifying, by the tissue perfusion monitoring computing device, one or more regions in the tissue region with altered perfusion states based on the contrast in the image data.

In another embodiment, the plurality of wavelengths are in the visible near infrared (VIS-NIR) or shortwave infrared (SWIR) regions.

In another embodiment, the image data is hyperspectral image data.

In another embodiment, analyzing the image data further comprises: generating, by the tissue perfusion monitoring computing device, hypercubes based on the collected hyperspectral image data; and analyzing, by the tissue perfusion monitoring computing device, the hypercubes to identify one or more of the plurality of wavelengths resulting in contrast in the hyperspectral image data.

In another embodiment, the method further comprises: generating, by the tissue perfusion monitoring computing device, a score video to monitor the one or more regions in the tissue region with the altered perfusion states over time.

In another embodiment, the method further comprises: identifying, by the tissue perfusion monitoring computing device, a hypoperfusion state based on the generated score videos.

In another embodiment, the image data is collected using a dual polarization architecture.

In another embodiment, the hyperspectral image data is collected in real time.

In one embodiment, there is a tissue perfusion monitoring computing device comprising: a non-transitory memory comprising programmed instructions stored thereon for detecting tissue perfusion; and one or more processors coupled to the memory and configured to execute the stored programmed instructions to: collect image data from an image sensor positioned to collect interacted photons from a tissue region resulting from illumination of the tissue sample at a plurality of wavelengths, analyze the image data to identify one or more of the plurality of wavelengths resulting in contrast in the image data, and identify one or more regions in the tissue region with altered perfusion states based on the contrast in the image data.

In another embodiment, the plurality of wavelengths are in the visible near infrared (VIS-NIR) or shortwave infrared (SWIR) regions.

In another embodiment, the image data is hyperspectral image data.

In another embodiment, analyzing the image data further comprises: generating hypercubes based on the collected hyperspectral image data; and analyzing the hypercubes to identify one or more of the plurality of wavelengths resulting in contrast in the hyperspectral image data.

In another embodiment, the processor further generates, based on the stored programmed instructions, a score video to monitor the one or more regions in the tissue region with the altered perfusion states over time.

In another embodiment, the processor further identifies, based on the stored programmed instructions, a hypoperfusion state based on the generated score video.

In another embodiment, the processor collects the image data using a dual polarization architecture.

In another embodiment, the processor collects the image data in real time.

In one embodiment, there is a non-transitory computer readable medium having stored thereon instructions for detecting tissue perfusion that when executed by one or more processors, causes the one or more processors to: collect image data from an image sensor positioned to collect interacted photons from a tissue region resulting from illumination of the tissue sample at a plurality of wavelengths; analyze the image data to identify one or more of the plurality of wavelengths resulting in contrast in the image data; and identify one or more regions in the tissue region with altered perfusion states based on the contrast in the image data.

In another embodiment, the plurality of wavelengths are in the visible near infrared (VIS-NIR) or shortwave infrared (SWIR) regions.

In another embodiment, the image data is hyperspectral image data.

In another embodiment, the instructions, when executed by the one or more processors, further cause the one or more processors in the analyze step to: generate hypercubes based on the collected hyperspectral image data; and analyze the hypercubes to identify one or more of the plurality of wavelengths resulting in contrast in the hyperspectral image data.

In another embodiment, the instructions, when executed by the one or more processors, further cause the one or more processors to generate a score video to monitor the one or more regions in the tissue region with the altered perfusion states over time.

In another embodiment, the instructions, when executed by the one or more processors, further cause the one or more processors to identify a hypoperfusion state based on the generated score videos.

In another embodiment, the image data is collected using a dual polarization architecture.

In another embodiment, the hyperspectral image data is collected in real time.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts a block diagram of an illustrative environment with an exemplary tissue perfusion monitoring computing device.

FIG. 2 depicts a block diagram of the exemplary tissue perfusion monitoring computing device of FIG. 1.

FIG. 3 depicts a flowchart of an illustrative method for improved tissue perfusion monitoring;

FIG. 4 depicts illustrative sets of score images obtained to monitor blood oxygenation before deoxygenation, during deoxygenation, and after reperfusion obtained using VIS-NIR imaging.

FIG. 5 depicts an illustrative set of score images obtained to monitor blood oxygenation before deoxygenation, during deoxygenation, and after reperfusion obtained using SWIR imaging.

FIG. 6 depicts illustrative sets of score images obtained to monitor blood oxygenation before deoxygenation, after one minute of deoxygenation, and after five minutes of deoxygenation obtained using a dual-polarization VIS-NIR platform.

FIG. 7 illustrates in vivo imaging results for a perfused porcine bowel model. FIG. 7 illustrates the detection of perfused bowel tissue from ischemic bowel tissue.

FIG. 8 illustrates in vivo imaging results for the porcine bowel model that was restricted to induce ischemia. FIG. 8 illustrates the detection of ischemic bowel tissue from perfused bowel tissue.

FIG. 9 illustrates a graph of the perfusion score over time for the perfused bowel region of FIG. 8 versus the ischemic bowel region of FIG. 8. The ischemic bowel region has a higher score than the perfused bowel region because the score image is used to detect ischemic bowel tissue.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

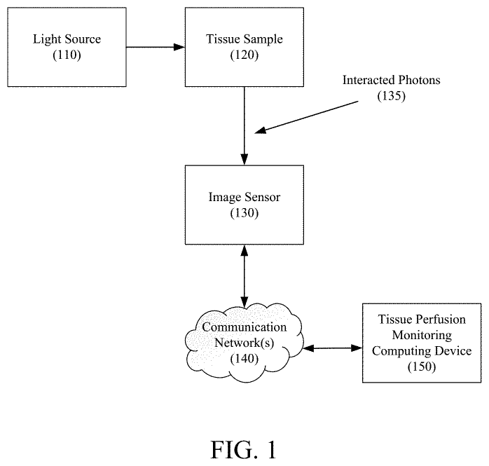

Referring to FIG. 1, an illustrative environment with an exemplary tissue perfusion monitoring computing device is depicted. The environment includes at least one light source 110 configured to generate photons to illuminate a tissue 120, an image sensor 130 positioned to collect interacted photons 135, and a tissue perfusion monitoring computing device 150 coupled to the image sensor via one or more communication networks 140, although the environment can include other types and/or numbers of devices or systems coupled in other manners, such as additional server devices. This technology provides a number of advantages including providing methods, non-transitory computer readable media, and tissue perfusion monitoring computing devices that provide improved tissue perfusion monitoring. In particular, certain implementations of this technology provide a real-time, reagentless, and non-contact method for monitoring tissue perfusion during surgical procedures in order to increase the effectiveness of hemodynamic protocols.

### Light Source

In an embodiment, at least one light source 110 generates photons that are directed to a tissue 120 in a human or animal. The at least one light source 110 is not limited and can be any light source that is useful in providing illumination. In an embodiment, the at least one light source 110 may be used in concert with or attached to endoscope. Other ancillary requirements, such as power consumption, emitted spectra, packaging, thermal output, and so forth may be determined based on the particular application that the at least one light source 110 is used. In some embodiments, the at least one light source 110 is a light element, which is an individual device that emits light. The light elements are not limited and may include an incandescent lamp, halogen lamp, light emitting diode (LED), chemical laser, solid state laser, organic light emitting diode (OLED), electroluminescent device, fluorescent light, gas discharge lamp, metal halide lamp, xenon arc lamp, induction lamp, or any combination of these light sources. In other embodiments, the at least one light source 110 is a light array, which is a grouping or assembly of more than one light element placed in proximity to each other.

In some embodiments, the at least one light source 110 has a particular wavelength that is intrinsic to the light element or to the light array. In other embodiments, the wavelength of a light source 110 may be modified by filtering or tuning the photons that are emitted by the light source. In still other embodiments, a plurality of light sources 110 having different wavelengths are combined. In one embodiment, the selected wavelength of the at least one light source 110 is in the visible-near infrared (VIS-NIR) or shortwave infrared (SWIR) ranges. These correspond to wavelengths of about 400 nm to about 1100 nm (VIS-NIR) or about 850 nm to about 1800 nm (SWIR). The above ranges may be used alone or in combination of any of the listed ranges. Such combinations include adjacent (contiguous) ranges, overlapping ranges, and ranges that do not overlap.

In some embodiments, the at least one light source 110 comprises a modulated light source. The choice of modulated light source 110 and the techniques of modulating the light source are not limited. In some embodiments, the modulated light source 110 is one or more of a filtered incandescent lamp, filtered halogen lamp, tunable LED array, tunable solid state laser array, tunable OLED array, tunable electroluminescent device, filtered fluorescent light, filtered gas discharge lamp, filtered metal halide lamp, filtered xenon arc lamp, filtered induction lamp, or any combination of these light sources. In some embodiments, tuning is accomplished by increasing or decreasing the intensity or duration at which the individual light elements 110 are powered. Alternatively, tuning is accomplished by a fixed or tunable filter that filters light emitted by the individual light elements. In still other embodiments, at least one light source 110 is not tunable. A light source 110 that is not tunable cannot change its emitted light spectra, but it can be turned on and off by the appropriate controls.

Imaging is performed by filtering and detecting interacted photons 135 that are reflected from the body of the human or animal patient 120 using the image sensor 130 and associated optics, such as filters. The image sensor 130 can be any suitable image sensor for molecular chemical imaging (MCI). The techniques and devices for filtering are not limited and include any of fixed filters, multi-conjugate filters, and conformal filters. In fixed filters, the functionality of the filter cannot be changed, though the filtering can be changed by mechanically moving the filter into or out of the light path. In some embodiments, real-time image detection is employed using a dual polarization configuration using either multi-conjugate filters or conformal filters. In some embodiments, the filter is a tunable filter that comprises a multi-conjugate filter. The multi-conjugate filter is an imaging filter with serial stages along an optical path in a Solc filter configuration. In such filters, angularly distributed retarder elements of equal birefringence are stacked in each stage with a polarizer between stages.

A conformal filter can filter a broadband spectra into one or more passbands. Example conformal filters include a liquid crystal tunable filter, an acousto-optical tunable filter, a Lyot liquid crystal tunable filter, an Evans Split-Element liquid crystal tunable filter, a Solc liquid crystal tunable filter, a Ferroelectric liquid crystal tunable filter, a Fabry Perot liquid crystal tunable filter, and combinations thereof.

In an embodiment, the image is collected by an image sensor 130 that is a camera chip 130. The camera chip 130 is not limited, but in some embodiments is selected depending on the expected spectra that is reflected from the skin, tissues, or organs of the human or animal patient. In some embodiments, the camera chip 130 is one or more of a charge coupled device (CCD), a complementary metal oxide semiconductor (CMOS), an indium gallium arsenide (InGaAs) camera chip, a platinum silicide (PtSi) camera chip, an indium antimonide (InSb) camera chip, a mercury cadmium telluride (HgCdTe) camera chip, or a colloidal quantum dot (CQD) camera chip. In some embodiments, each or the combination of the above-listed camera chips 130 is a focal plane array (FPA). In some embodiments, each of the above-identified camera chips 130 includes quantum dots to tune their bandgaps, thereby altering or expanding sensitivity to different wavelengths. The visualization techniques are not limited, and include one or more of VIS, NIR, SWIR, autofluorescence, or Raman spectroscopy. Although the image sensor 130 is illustrated as a standalone device, the image sensor could be incorporated in the tissue perfusion monitoring computing device 150 or in a device with the at least one light source 110.

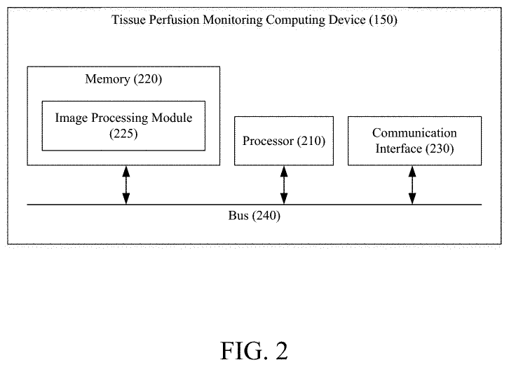

Referring to FIGS. 1-2, the tissue perfusion monitoring computing device 150 in this example includes one or more processors 210, a memory 220, and/or a communication interface 230, which are coupled together by a bus 240 or other communication link, although the tissue perfusion monitoring computing device 150 can include other types and/or numbers of elements in other configurations. The one or more processors 210 of the tissue perfusion monitoring computing device 150 may execute programmed instructions stored in the memory 220 for the any number of the functions described and illustrated herein. The one or more processors 210 of the tissue perfusion monitoring computing device 150 may include one or more CPUs or general purpose processors with one or more processing cores, for example, although other types of processors can also be used.

The memory 220 of the tissue perfusion monitoring computing device 150 stores these programmed instructions for one or more aspects of the present technology as described and illustrated herein, although some or all of the programmed instructions could be stored elsewhere. A variety of different types of memory storage devices 220, such as random access memory (RAM), read only memory (ROM), hard disk, solid state drives, flash memory, or other computer readable medium which is read from and written to by a magnetic, optical, or other reading and writing system that is coupled to the one or more processors, can be used for the memory.

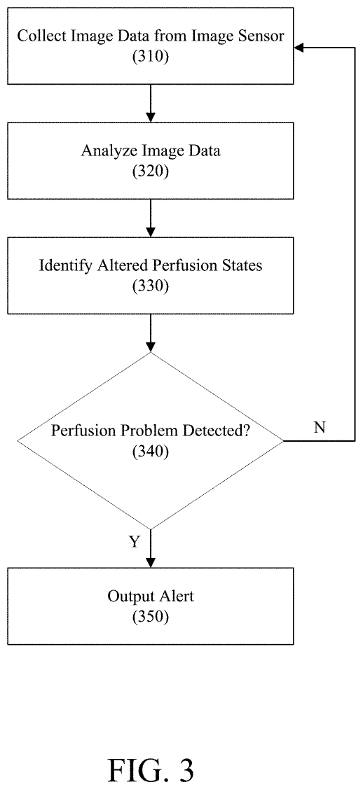

Accordingly, the memory 220 of the tissue perfusion monitoring computing device 150 can store one or more applications that can include executable instructions that, when executed by the one or more processors 210, cause the tissue perfusion monitoring computing device to perform actions, such as to perform the actions described and illustrated below with reference to FIG. 3. In some embodiments, the one or more applications can be implemented as modules or components of one or more other applications. In some embodiments, the one or more applications can be implemented as operating system extensions, module, plugins, or the like.

In some embodiments, the one or more applications may be operative in a cloud-based computing environment. In some embodiments, the one or more applications can be executed within or as one or more virtual machines or one or more virtual servers that may be managed in a cloud-based computing environment. In some embodiments, the one or more applications and/or the tissue perfusion monitoring computing device 150 may be located in one or more virtual servers running in a cloud-based computing environment rather than being tied to one or more specific physical network computing devices. In some embodiments, the one or more applications may run in one or more virtual machines (VMs) executing on the tissue perfusion monitoring computing device 150. Additionally, in one or more embodiments of this technology, one or more virtual machines running on the tissue perfusion monitoring computing device 150 may be managed or supervised by a hypervisor.

In a particular embodiment, the memory 220 of the tissue perfusion monitoring computing device 150 includes an image processing module 225, although the memory can include other policies, modules, databases, or applications, for example. The image processing module 225 may be configured to analyze image data from the image sensor 130 to determine tissue perfusion values of the illuminated tissue 120 based on the image data, although the image processing module could perform other functions. By way of example only, the image processing module 225 may apply one or more machine learning techniques such as an image-weighted Bayesian function, logistic regression, linear regression, regression with regularization, partial least squares regression (PLSR), partial least squares discriminant analysis (PLSDA), naïve Bayes, classification and regression trees (CART), support vector machines, or a neural network to process the image data.

The communication interface 230 of the tissue perfusion monitoring computing device 150 operatively couples and communicates between the tissue perfusion monitoring computing device, the image sensor 130, the additional sensors, the client devices and/or the server devices, which are all coupled together by the one or more illustrated communication networks 140. Other types and/or numbers of communication networks 140 or systems with other types and/or numbers of connections and/or configurations to other devices and/or elements can also be used.

By way of example only, the communication network(s) 140 shown in FIG. 1 can include one or more local area networks (LANs) and/or one or more wide area networks (WANs), and can use TCP/IP over Ethernet and industry-standard protocols, although other types and/or numbers of protocols and/or communication networks can be used. The one or more communication networks 140 in this example can employ any suitable interface mechanisms and network communication technologies including, for example, teletraffic in any suitable form (e.g., voice, modem, and the like), Public Switched Telephone Networks (PSTNs), Ethernet-based Packet Data Networks (PDNs), combinations thereof, and the like.

The tissue perfusion monitoring computing device 150 can be a standalone device or integrated with one or more other devices or apparatuses, such as, for example, the image sensor 130, one or more of the server devices, or one or more of the client devices. In particular embodiments, the tissue perfusion monitoring computing device 150 can include or be hosted by one of the server devices or one of the client devices. Other arrangements are also possible.

Although the exemplary environment with the tissue perfusion monitoring computing device 150, light source 110, image sensor 130, and one or more communication networks 140 are described and illustrated herein, other types and/or numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

One or more of the devices depicted in the environment, such as, for example, the tissue perfusion monitoring computing device 150 may be configured to operate as virtual instances on the same physical machine. In other words, one or more of the tissue perfusion monitoring computing device 150, client devices, or server devices may operate on the same physical device rather than as separate devices communicating through one or more communication networks 140. Additionally, there may be more or fewer tissue perfusion monitoring computing devices 150 than illustrated in FIG. 1.

In some embodiments, a plurality of computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on one or more computer systems that extend across any suitable network using any suitable interface mechanisms and traffic technologies, including, by way of example only, wireless networks, cellular networks, PDNs, the Internet, intranets, and combinations thereof.

The examples may also be embodied as one or more non-transitory computer readable media (e.g., the memory 220) having instructions stored thereon for one or more aspects of the present technology as described and illustrated by way of the examples herein. The instructions in some examples include executable code that, when executed by one or more processors (e.g., the one or more processors 210), cause the one or more processors to carry out steps necessary to implement the methods of the examples of this technology that are described and illustrated herein.

An exemplary method of tissue perfusion monitoring will now be described with reference to FIG. 3. As shown in FIG. 3, the tissue perfusion monitoring computing device may collect 310 image data from the image sensor. The image data can be hyperspectral image data, for example. In some embodiments, the image sensor is positioned to collect 310 interacted photons from a tissue region resulting from illumination of the tissue sample at a plurality of wavelengths using the light source. In some embodiments, the light source is located on an endoscopic device. In some embodiments, the light source illuminates the tissue region using wavelengths in the visible near infrared (VIS-NIR) and/or shortwave infrared (SWIR) regions.

The tissue perfusion monitoring computing device may analyze 320 the image data to identify one or more of the plurality of wavelengths resulting in contrast in the image data. In some embodiments, the tissue perfusion monitoring computing device generates hypercubes based on collected hyperspectral image data. The hypercubes may be analyzed 320 to identify one or more of the plurality of wavelengths resulting in contrast in the hyperspectral image data.

The tissue perfusion monitoring computing device may identify 330 one or more regions in the tissue region with altered perfusion states based on the contrast in the image data. In some embodiments, the tissue perfusion monitoring computing device may monitor the altered perfusion states over time. In some embodiments, the tissue perfusion monitoring computing device may monitor perfusion issues, such as during a surgical procedure. For example, the tissue perfusion monitoring computing device may monitor perfusion to identify whether a hypoperfusion state occurs. If a hypoperfusion state is identified 340, an alert may be signaled 350 so that one or more hemodynamic protocols may be altered. In some embodiments, the tissue perfusion monitoring computing device may be used to discriminate between tissue undergoing ischemia and normally perfused tissue. In some embodiments, the tissue perfusion monitoring computing device may be used to discriminate between tissue oxygenation saturation levels. In another embodiment, the tissue perfusion monitoring computing device may be used to monitor pulse oximetry in a non-contact manner using the image data. In one embodiment, the tissue perfusion monitoring computing device generates score images and/or a score video to monitor the one or more regions in the tissue region with the altered perfusion states over time.

With this technology, tissue perfusion and blood oxygenation can be monitored in a non-contact, reagentless manner. For example, the technology can advantageously be used during surgical procedures in a non-invasive manner to allow for the adjustment of hemodynamic protocols based on changes in perfusion states for the patient. In certain embodiments, the technology can be used for improved planning of the above described surgical procedures.

In alternate embodiments, the technology may be used to identify trauma victims, such as wounded soldiers, who are hemorrhaging in the field prior to being transported to a medical treatment facility. Hemorrhage has been identified as a primary cause of death for combat injuries that are potentially survivable if treatment were provided before reaching a medical treatment facility. Enhancing medic capabilities with multi-modal equipment could lead to increased survivability of trauma wounds.

In some embodiments, a tissue/trauma/triage sensor (“3TS”) system may incorporate molecular chemical imaging, intraoperative imaging, tissue identification, and advanced visualization principles to provide real-time or near real-time assessment of trauma victims in a field surgical (i.e., non-hospital) setting. In some embodiments, a 3TS system may be used to image subcutaneous vasculature for improved peripheral venous access, particularly in volume-depleted patients. In such embodiments, the 3TS system may enable field medical personnel or first responders to determine appropriate venous access in lieu of placing a tourniquet and performing visual palpitations.

In some embodiments, a 3TS system may be used to evaluate tissue oxygenation levels to enable determination of a patient's oxygen needs, yield a measure of tissue viability, and provide a guide for tissue debridement. In such embodiments, a 3TS system may provide a localized, tissue-specific oxygen saturation reading (instead of a global reading provided by pulse oximeters). As a result, an assessment of tissue viability and guidance for tissue debridement may represent a significant advance over the current standard of care (i.e., visual observation).

In some embodiments, a 3TS system may be used to estimate blood pressure levels without contacting the patient by correlating perfusion with blood pressure. Use of a 3TS system has further significant advantages of being both hands-free and being capable of use in a complex acoustic environment, such as aboard a helicopter, where a conventional blood pressure cuff used with a stethoscope is ineffective.

In some embodiments, a 3TS system may include a VIS-NIR multi-conjugate imaging modality and/or a SWIR multi-conjugate imaging modality. Visible and NIR spectroscopic methods may be used to visualize blood vessels, to distinguish veins from arteries and surrounding tissues, and/or to improve access to peripheral veins. SWIR spectroscopic methods may be used to enhance sensitivity to chromophores, such as lipids, enable characterization of various bodily conditions, such as bruising, atherosclerotic plaque, cancer, and burns, and visualize changes in vasculature and collagen structure.

In some embodiments, a 3TS system may further include a heads up display (HUD), which may be incorporated into a pair of glasses or a helmet, to enable an augmented reality display of the vasculature of a patient or trauma victim. In some embodiments, a 3TS system may further include a handheld device used to image the vasculature of the patient or trauma victim.

### EXAMPLES

### Example 1—VIS-NIR Imaging of Tissue Deoxygenation and Reperfusion

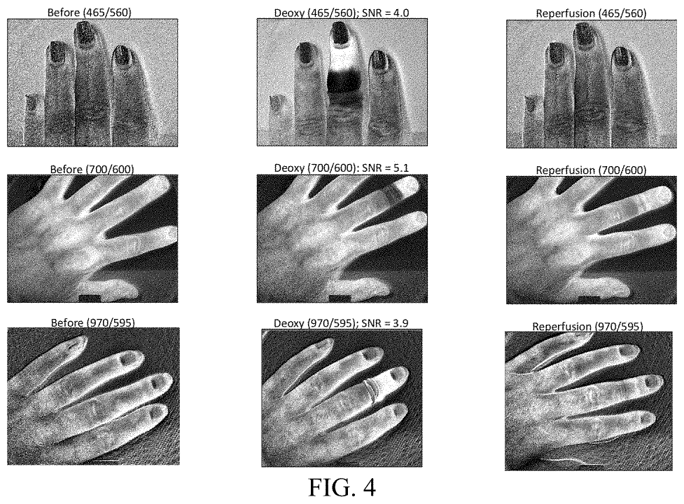

Molecular chemical imaging was utilized to demonstrate visualization of tissue oxygenation in the visible and near infrared spectral regions. Blood flow was temporarily restricted to the top of the subjects' fingers with a rubber band. VIS-NIR imaging was then performed on the subjects' hands to obtain score images as shown in FIG. 4, which illustrates three subjects' hands. The images from left to right show the hands before deoxygenation, during deoxygenation, and after reperfusion. The results illustrate that the tissue regions suffering restricted blood flow show spectra response characteristic of Hb compared with regions that are not blood restricted. Further, grades of oxygenation are identifiable. Thus, the imaging provided the ability to different between oxygenated and deoxygenated regions in the subjects' fingers.

### Example 2—SWIR Imaging of Tissue Deoxygenation and Reperfusion

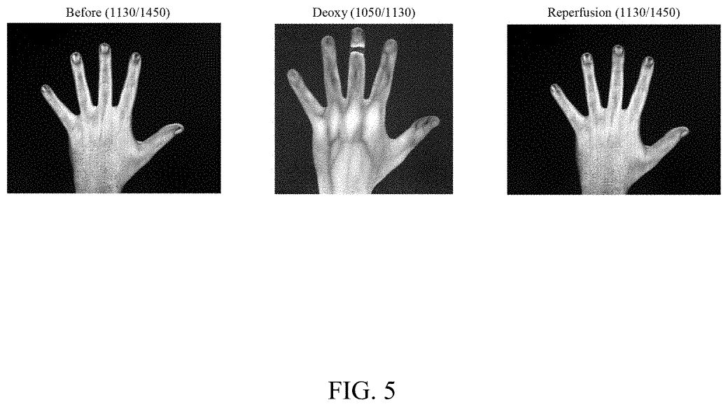

Molecular chemical imaging was utilized to demonstrate visualization of tissue oxygenation in the shortwave infrared spectral region. Blood flow was temporarily restricted to the top of the subject's fingers with a rubber band. SWIR imaging was then performed on the subject's hand to obtain score images as shown in FIG. 5. The images in FIG. 5 from left to right show the hand before deoxygenation, during deoxygenation, and after reperfusion. The imaging provided the ability to different between oxygenated and deoxygenated regions in the subject's finger.

### Example 3—Dual Polarization VIS-NIR Imaging of Tissue Deoxygenation

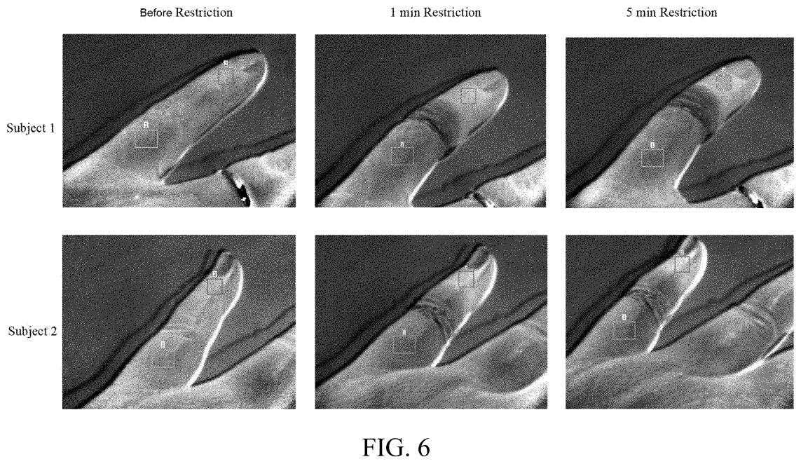

Molecular chemical imaging was utilized to demonstrate visualization of tissue oxygenation using a dual polarization VIS-NIR platform. Blood flow was temporarily restricted to the top of the subjects' fingers with a rubber band. VIS-NIR imaging was then performed on the subjects' hands to obtain score images as shown in FIG. 6. The images in FIG. 6 show, from left to right, the subjects' hands before restriction, after one minute of restriction, and after five minutes of restriction. The imaging provided the ability to differentiate between oxygenated and deoxygenated regions in the subjects' fingers and the amount of deoxygenation over time.

### Example 4—Dual Polarization VIS-NIR Imaging of Porcine Bowel

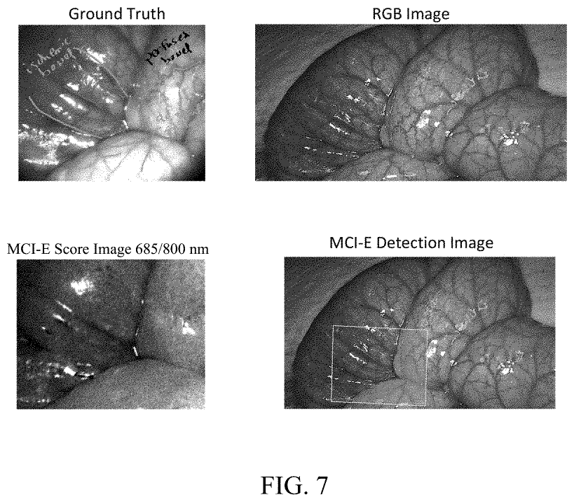

Dual polarization molecular chemical imaging was utilized to visualize in vivo perfused porcine small bowel in the presence of ischemic small bowel. The Ground Truth image in FIG. 7 (top left) shows where in the image the ischemic and perfused bowel sections are; the RGB image depicts what the human eye sees when comparing these two tissues, and the MCI-E Detection image shows the detection of perfused tissue (in green). This capability would be useful for monitoring tissue viability, for example, in real time during intraoperative procedures.

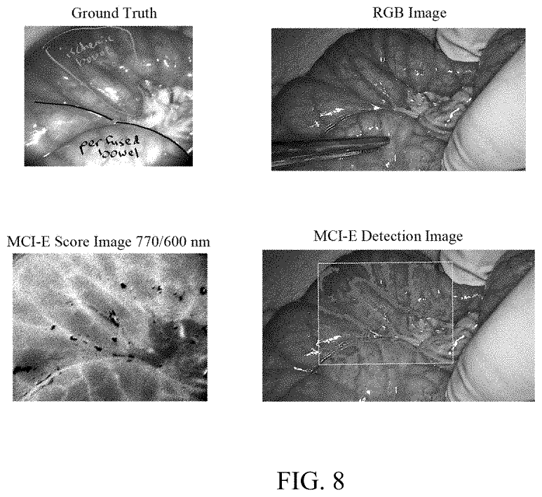

In FIG. 8, the bowel blood vessels are targeted using a different set of wavelengths. Blood vessel detections are shown in green in the MCI-E Detection image (lower right).

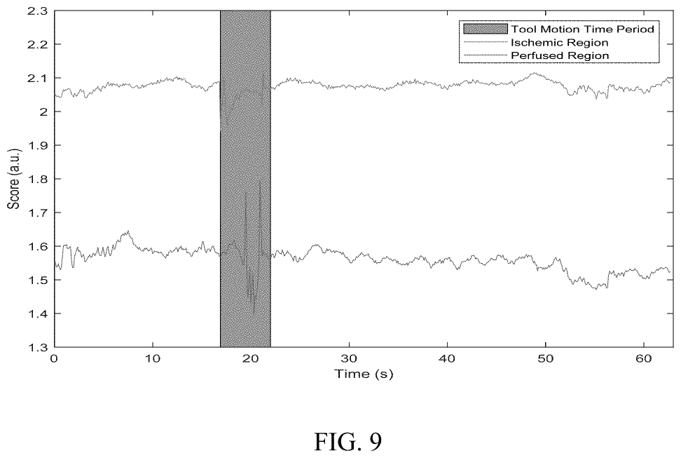

In FIG. 9, the score of an average region of perfused bowel is plotted over time, along with the score of an average region of ischemic bowel. The ischemic bowel region has a higher score than the perfused bowel region because the score image is used to detect ischemic bowel tissue. The gray region indicates a time period when a tool was in the field of view creating additional motion. Fourier analysis is a method which may help to evaluate such features as respiration or hemodynamic (blood flow) signatures associated with heart rate. Fourier analysis on the time series could be used to analyze the periodicities observed during real-time imaging of the ischemic and perfused porcine bowel regions, as well as other scores. The periodicity in the blue lower plot of the perfused bowel may be due to motion from breathing.

In the above detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of detecting tissue perfusion, the method comprising:
collecting, by a tissue perfusion monitoring computing device, image data from an image sensor positioned to collect interacted photons from a tissue region resulting from illumination of the tissue sample at a plurality of wavelengths;
analyzing, by the tissue perfusion monitoring computing device, the image data to identify one or more of the plurality of wavelengths resulting in contrast in the image data; and
identifying, by the tissue perfusion monitoring computing device, one or more regions in the tissue region with altered perfusion states based on the contrast in the image data.

2. The method of claim 1, wherein the plurality of wavelengths are in the visible near infrared (VIS-NIR) or shortwave infrared (SWIR) regions.

3. The method of claim 1, wherein the image data is hyperspectral image data.

4. The method of claim 3, wherein analyzing the image data further comprises:
generating, by the tissue perfusion monitoring computing device, hypercubes based on the collected hyperspectral image data; and
analyzing, by the tissue perfusion monitoring computing device, the hypercubes to identify one or more of the plurality of wavelengths resulting in contrast in the hyperspectral image data.

5. The method of claim 1, further comprising:
generating, by the tissue perfusion monitoring computing device, a score video to monitor the one or more regions in the tissue region with the altered perfusion states over time.

6. The method of claim 5, further comprising:
identifying, by the tissue perfusion monitoring computing device, a hypoperfusion state based on the generated score videos.

7. The method of claim 1, wherein the image data is collected using a dual polarization architecture.

8. The method of claim 7, wherein the hyperspectral image data is collected in real time.

9. A tissue perfusion monitoring computing device comprising:
a non-transitory memory comprising programmed instructions stored thereon for detecting tissue perfusion; and
one or more processors coupled to the memory and configured to execute the stored programmed instructions to:
collect image data from an image sensor positioned to collect interacted photons from a tissue region resulting from illumination of the tissue sample at a plurality of wavelengths,
analyze the image data to identify one or more of the plurality of wavelengths resulting in contrast in the image data, and
identify one or more regions in the tissue region with altered perfusion states based on the contrast in the image data.

10. The tissue perfusion monitoring computing device of claim 9, wherein the plurality of wavelengths are in the visible near infrared (VIS-NIR) or shortwave infrared (SWIR) regions.

11. The tissue perfusion monitoring computing device of claim 9, wherein the image data is hyperspectral image data.

12. The tissue perfusion monitoring computing device of claim 11, wherein the analyzing the image data further comprises:
generating hypercubes based on the collected hyperspectral image data; and
analyzing the hypercubes to identify one or more of the plurality of wavelengths resulting in contrast in the hyperspectral image data.

13. The tissue perfusion monitoring computing device of claim 9, wherein the processor further generates, based on the stored programmed instructions, a score video to monitor the one or more regions in the tissue region with the altered perfusion states over time.

14. The tissue perfusion monitoring computing device of claim 13, wherein the processor further identifies, based on the stored programmed instructions, a hypoperfusion state based on the generated score video.

15. The tissue perfusion monitoring computing device of claim 9, wherein the processor collects the image data using a dual polarization architecture.

16. The tissue perfusion monitoring computing device of claim 15, wherein the processor collects the image data in real time.

17. A non-transitory computer readable medium having stored thereon instructions for detecting tissue perfusion that when executed by one or more processors, causes the one or more processors to:
collect image data from an image sensor positioned to collect interacted photons from a tissue region resulting from illumination of the tissue sample at a plurality of wavelengths;
analyze the image data to identify one or more of the plurality of wavelengths resulting in contrast in the image data; and
identify one or more regions in the tissue region with altered perfusion states based on the contrast in the image data.

18. The non-transitory computer readable medium of claim 17, wherein the plurality of wavelengths are in the visible near infrared (VIS-NIR) or shortwave infrared (SWIR) regions.

19. The non-transitory computer readable medium of claim 17, wherein the image data is hyperspectral image data.

20. The non-transitory computer readable medium of claim 19, wherein the instructions, when executed by the one or more processors, further cause the one or more processors in the analyze step to:
generate hypercubes based on the collected hyperspectral image data; and
analyze the hypercubes to identify one or more of the plurality of wavelengths resulting in contrast in the hyperspectral image data.

21. The non-transitory computer readable medium of claim 17, wherein the instructions, when executed by the one or more processors, further cause the one or more processors to generate a score video to monitor the one or more regions in the tissue region with the altered perfusion states over time.

22. The non-transitory computer readable medium of claim 21, wherein the instructions, when executed by the one or more processors, further cause the one or more processors to identify a hypoperfusion state based on the generated score videos.

23. The non-transitory computer readable medium of claim 17, wherein the image data is collected using a dual polarization architecture.

24. The non-transitory computer readable medium of claim 23, wherein the hyperspectral image data is collected in real time.
